(19) United States
(12) Patent Application Publication
(10) Pub. No.: US 2025/0035532 A1
(43) Pub. Date: Jan. 30, 2025

(54) METHODOLOGY TO CHARACTERIZE EMPTY-FULL RATIO IN CRUDE AAV SAMPLES

(71) Applicant: Regeneron Pharmaceuticals, Inc., Tarrytown, NY (US)

(72) Inventors: Deepanshu Choudhary, New Rochelle, NY (US); Michael Rosconi, New City, NY (US); Erica Pyles, New City, NY (US)

(21) Appl. No.: 18/786,335

(22) Filed: Jul. 26, 2024

Related U.S. Application Data
(60) Provisional application No. 63/529,036, filed on Jul. 26, 2023.

(51) Int. Cl. G01N 15/04; B01D 15/38; C12N 5/071; C12N 7/00; G01N 1/40

(52) U.S. Cl. CPC G01N 15/042; B01D 15/3809; C12N 5/0686; C12N 7/00; G01N 1/4077; C12N 2750/14143; C12N 2750/14151; G01N 2333/015

(57) ABSTRACT

The present invention provides methods to identify and quantify empty, partial and full capsids. The capsids can be purified from a crude AAV sample using affinity chromatography and eluted from the solid support. The purified capsids can then be subjected to density gradient equilibrium analytical ultracentrifugation (DGE-AUC) for characterization and quantitation of capsid content.

### CROSS REFERENCE TO RELATED APPLICATIONS

This application claims the benefit of priority to U.S. Provisional Application No. 63/529,036, filed Jul. 26, 2023, which is incorporated herein by reference in its entirety.

### FIELD

The present invention generally pertains to methods for identifying and quantifying capsid content in a crude AAV sample to monitor and ensure the quality of AAV production.

### BACKGROUND

Gene therapy biopharmaceuticals mediate therapeutic effects by transcription and/or translation of transferred genetic material, such as integrating genetic material into the host genome, and are used to treat, prevent or cure a disease. Currently, gene therapy is one of the most investigated therapeutic modalities in preclinical and clinical settings. However, gene therapy experienced a major setback in the late 1990's and early 2000's which raised concerns about the safety of gene therapy and highlighted the critical need for safer gene delivery vectors. A better understanding of gene delivery vectors and advancing the manufacture of safe and effective vectors is necessary to mitigate safety risks.

Gene delivery vectors are essential to ensure efficient gene delivery to the target tissue and cells. The ideal gene delivery system should have high gene transfer efficiency, low toxicity to the cell, and single cell specificity to the intended target. Based on gene delivery vector types, vectors can be divided into non-viral vectors and viral vectors. Due to the high gene transfer efficiency of viral vectors, they have been widely used in clinical trials.

Adeno-associated virus (AAV) is the most widely used viral vector for in vivo gene therapy applications. AAVs have low immunogenicity and they can enable long-term, stable gene expression. The use of AAVs for gene therapy has created the need for analytical methods to monitor and characterize these products. Process related and product related impurities should be monitored to ensure product quality and process consistency.

Biopharmaceutical products must meet very high standards of purity. Thus, it is important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, an inherent characteristic of the AAV manufacturing process is the production of empty capsids, a capsid which lacks any packaged transgene. The presence of empty capsids in drug products should be monitored and can be unacceptable above a certain threshold. Sometimes, even empty capsids could result in an unwanted immune response and may reduce the potency of the gene therapy drug.

It will be appreciated that a need exists for methods to characterize capsid content in a drug substance or other product to mitigate safety risks.

### SUMMARY

This disclosure provides methods of methods of characterizing at least one AAV vector in a sample containing at least one impurity in a sample. In some exemplary embodiments, the method comprises: (a) subjecting a sample containing at least one AAV vector to affinity purification to produce an enriched sample; (b) contacting said enriched sample to a salt solution to form a homogeneous solution; and (c) subjecting said homogeneous solution to density gradient equilibrium analytical ultracentrifugation (DGE-AUC) to characterize the at least one AAV vector.

In one aspect, the affinity purification comprises affinity chromatography.

In one aspect, the affinity purification is a small-scale affinity purification.

In one aspect, the affinity purification occurs in a medium throughput or semi-high throughput manner.

In one aspect, the affinity purification comprises contacting said sample to a resin conjugated to an affinity ligand.

In one aspect, the affinity ligand comprises an antigen-binding protein, an antibody, a variant thereof, or a fragment thereof.

In one aspect, the resin is POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, or a combination thereof.

In one aspect, the method includes subjecting the resin to at least one wash step, at least two wash steps, at least three wash steps, or at least four wash steps.

In one aspect, a wash buffer used in the wash step(s) comprises about 1×TBS (20 mM Tris pH 7.5, 150 mM NaCl), about 2×TBS, about 1×TBS with 20% ETOH, about 1×PBS (137 mM NaCl, 10 mM phosphate, 2.7 mM KCl, pH 7.4), about 2×PBS, about 1×PBS with 18% EtOH, or combinations thereof.

In one aspect, the affinity purification comprises an elution step.

In one aspect, the elution step includes an elution buffer, optionally wherein said elution buffer comprises glycine, poloxamer 188, or a combination thereof. In a specific aspect, the elution buffer comprises about 50 mM glycine and about 0.01% poloxamer 188 at a pH of about 3. In another specific aspect, the elution buffer comprises about 100 mM glycine and about 0.01% poloxamer 188 at a pH of about 2.

In one aspect, the salt solution includes cesium chloride, cesium sulfate, cesium bromide, cesium formate, cesium acetate, cesium iodide, cesium selenite, potassium bromide, rubidium bromide, rubidium chloride, cesium TFA, sodium iothalamate, or a combination thereof.

In one aspect, the concentration of salt in said salt solution is about 1.30 g/mL to about 1.40 g/mL or about 1.32 g/mL to about 1.36 g/mL. In a specific aspect, the salt concentration is about 1.34 g/mL.

In one aspect, the rotor speed of the analytical ultracentrifuge is from 10 k to 60 k rpm. In a specific aspect, the rotor speed is about 40 k rpm.

In one aspect, the at least one AAV vector comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

In one aspect, the sample is a crude sample.

In one aspect, the sample comprises a harvest pool or a tangential flow filtration (TFF) pool.

In one aspect, the concentration of the at least one AAV vector in the sample is from 1×10¹⁰ to 1×10¹² cp/mL.

In one aspect, the method includes quantification of empty capsids, partially filled capsids, and/or full capsids of said at least one AAV vector.

In one aspect, the method includes quantifying a ratio of absorbance at 260 nm to absorbance at 280 nm wavelengths of AAV vector species separated by density gradient equilibrium analytical ultracentrifugation.

In one aspect, the method includes detecting and/or quantifying high molecular weight species.

This disclosure also provides a method of characterizing at least one AAV vector in a crude sample. In some exemplary embodiments, the method comprises: (a) subjecting a sample containing at least one AAV vector to affinity purification to produce an enriched sample; (b) contacting said enriched sample to a salt solution to form a homogeneous solution; and (c) subjecting said homogeneous solution to density gradient equilibrium analytical ultracentrifugation to characterize the at least one AAV vector.

In one aspect, the affinity purification comprises affinity chromatography.

In one aspect, the affinity purification is a small-scale affinity purification. In a specific aspect, the sample is less than 100 μL of a purified AAV sample. In another specific aspect, the sample is less than 1 mL of a TFF pool sample. In a further specific aspect, the sample is 10 mL or less of a harvest pool sample.

In one aspect, the affinity purification occurs in a medium throughput manner.

In one aspect, the affinity purification comprises contacting said sample to a resin conjugated to an affinity ligand. In a specific aspect, the affinity ligand comprises an antigen-binding protein, an antibody, a variant thereof, or a fragment thereof.

In one aspect, the resin is POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, or a combination thereof.

In one aspect, the method includes subjecting said resin to at least two wash steps, at least three wash steps, or at least four wash steps.

In one aspect, a wash buffer used in the wash step(s) comprises about 1×TBS, about 2×TBS, about 1×TBS with 20% EtOH, about 1×PBS, about 2×PBS, about 1×PBS with 18% EtOH, or combinations thereof.

In one aspect, the affinity purification comprises an elution step.

In one aspect, the elution step includes an elution buffer, optionally wherein said elution buffer comprises glycine, poloxamer 188, or a combination thereof. In a specific aspect, the elution buffer comprises about 50 mM glycine and about 0.01% poloxamer 188 at a pH of about 3. In another specific aspect, the elution buffer comprises about 100 mM glycine and about 0.01% poloxamer 188 at a pH of about 2.

In one aspect, the salt solution includes cesium chloride, cesium sulfate, cesium bromide, cesium formate, cesium acetate, cesium iodide, cesium selenite, potassium bromide, rubidium bromide, rubidium chloride, cesium TFA, sodium iothalamate, or a combination thereof.

In one aspect, the concentration of salt in said salt solution is about 1.30 g/mL to about 1.40 g/mL or about 1.32 g/mL to about 1.36 g/mL. In a specific aspect, the concentration of salt in the salt solution is about 1.34 g/mL.

In one aspect, the rotor speed of the analytical ultracentrifuge is from 10 k to 60 k rpm. In a specific aspect, the rotor speed is about 40 k rpm.

In one aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

In one aspect, the sample comprises a harvest pool or a tangential flow filtration pool.

In one aspect, the concentration of said at least one AAV vector in said sample is from 1×10¹⁰ to 1×10¹² cp/mL.

In one aspect, the method includes quantifying empty capsids, partially filled capsids, and/or full capsids of said at least one AAV vector.

In one aspect, the method includes quantifying a ratio of absorbance at 260 nm to absorbance at 280 nm wavelengths of AAV vector species separated by density gradient equilibrium analytical ultracentrifugation.

In one aspect, the method includes detecting and/or quantifying high molecular weight species.

This disclosure provides a method of characterizing a viral vector. In some exemplary embodiments, the method comprises (a) subjecting cells that recombinantly produce a viral vector to lysis to form a lysed sample; (b) subjecting said lysed sample to filtration to form a harvest pool; (c) subjecting said harvest pool to affinity purification to form an enriched viral vector sample; and (d) subjecting said enriched viral vector sample to density gradient equilibrium analytical ultracentrifugation to characterize said viral vector.

In one aspect, the affinity purification is a small-scale affinity purification. In a specific aspect, the sample is less than 100 μL of a purified AAV sample. In another specific aspect, the sample is less than 1 mL of a TFF pool sample. In a further specific aspect, the sample is 10 mL or less of a harvest pool sample.

In one aspect, the affinity purification occurs in a medium throughput manner.

In one aspect, the method includes subjecting said harvest pool to tangential flow filtration prior to step (c).

In one aspect, the affinity purification comprises affinity chromatography.

In one aspect, the affinity purification comprises contacting said sample to a resin conjugated to an affinity ligand.

In one aspect, the affinity ligand comprises an antigen-binding protein, an antibody, a variant thereof, or a fragment thereof.

In one aspect, the resin is POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, or a combination thereof.

In one aspect, the method includes subjecting said resin to at least one wash step, at least two wash steps, at least three wash steps or at least four wash steps. In a specific aspect, a wash buffer used in the wash step(s) comprises about 1×TBS, about 2×TBS, about 1×TBS with 20% ETOH, about 1×PBS, about 2×PBS, about 1×PBS with 18% EtOH, or combinations thereof.

In one aspect, the affinity purification comprises an elution step.

In one aspect, the elution step includes an elution buffer, optionally wherein said elution buffer comprises glycine, poloxamer 188, or a combination thereof. In a specific aspect, the elution buffer comprises about 50 mM glycine and about 0.01% poloxamer 188 at a pH of about 3. In another specific aspect, the elution buffer comprises about 100 mM glycine and about 0.01% poloxamer 188 at a pH of about 2.

In one aspect, the method includes contacting said enriched viral vector sample to a salt solution to form a homogeneous mixture prior to step (d).

In one aspect, the salt solution includes cesium chloride, cesium sulfate, cesium bromide, cesium formate, cesium acetate, cesium iodide, cesium selenite, potassium bromide, rubidium bromide, rubidium chloride, cesium TFA, sodium iothalamate, or a combination thereof.

In one aspect, the concentration of salt in said salt solution is about 1.30 g/mL to about 1.40 g/mL or about 1.32 g/mL to about 1.36 g/mL. In a specific aspect, the salt concentration is about 1.34 g/mL.

In one aspect, the rotor speed of the analytical ultracentrifuge is from 10 k to 60 k rpm. In a specific aspect, the rotor speed is about 40 k rpm.

In one aspect, the viral vector comprises an AAV vector. In a specific aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

In one aspect, the concentration of said at least one viral vector in said sample is from 1×10¹⁰ to 1×10¹² cp/mL.

In one aspect, the method includes quantifying empty capsids, partially filled capsids, and/or full capsids of said at least one viral vector.

In one aspect, the method includes quantifying a ratio of absorbance at 260 nm to absorbance at 280 nm wavelengths of viral vector species separated by density gradient equilibrium analytical ultracentrifugation.

In one aspect, the method includes detecting and/or quantifying high molecular weight species.

In one aspect, the cells comprise HEK293, HeLa or Chinese Hamster Ovary (CHO) cells.

In one aspect, the cells are HEK293 cells.

This disclosure provides a method for quantifying the capsid content of a viral vector. In some exemplary embodiments, the method comprises: (a) subjecting a sample including a viral vector to affinity purification to form an enriched sample; and (b) subjecting said enriched sample to density gradient equilibrium analytical ultracentrifugation to quantify the capsid content of said viral vector.

This disclosure provides a method for characterizing critical quality attributes of a viral vector. In some exemplary embodiments, the method comprises: (a) subjecting a sample including a viral vector to affinity purification to form an enriched sample; and (b) subjecting said enriched sample to density gradient equilibrium analytical ultracentrifugation to characterize critical quality attributes of said viral vector.

In one aspect, the critical quality attributes include the empty/full ratio of said viral vector, the empty/partially filled/full ratio of said viral vector, low molecular weight species of said viral vector, and/or high molecular weight species of said viral vector.

These, and other, aspects of the present invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present invention.

### BRIEF DESCRIPTION OF THE DRAWINGS

This patent or application contains at least one drawing executed in color. Copies of this patent or patent application publication with color drawings will be provided by the Office upon request and payment of the necessary fee.

FIG. 1 illustrates empty, partially filled, and full capsids, according to an exemplary embodiment.

FIG. 2A shows a quantification of empty, partially filled and full capsids for an AAV8 sample after sedimentation velocity analytical ultracentrifugation (SV-AUC), according to an exemplary embodiment.

FIG. 2B shows a quantification of empty, partially filled and full capsids for an AAV8 sample after DGE-AUC utilizing a cesium chloride gradient, according to an exemplary embodiment.

FIG. 2C shows a comparison of quantifications of empty, partially filled and full capsids for an AAV8 sample using SV-AUC and DGE-AUC, according to an exemplary embodiment.

FIG. 3A shows a quantification of empty, partially filled and full capsids for an AAV5 sample after SV-AUC, according to an exemplary embodiment.

FIG. 3B shows a quantification of empty, partially filled and full capsids for an AAV5 sample after DGE-AUC utilizing a cesium sulfate gradient, according to an exemplary embodiment.

FIG. 3C shows a comparison of quantifications of empty, partially filled and full capsids for an AAV5 sample using SV-AUC and DGE-AUC, according to an exemplary embodiment.

FIG. 4 shows DGE-AUC analysis of a crude TFF pool sample and highlights background signal from the sample, according to an exemplary embodiment.

FIG. 5 illustrates an affinity purification process for crude AAV samples, according to an exemplary embodiment.

FIG. 6 illustrates a workflow of method development, data collection, and data analysis for the method of the present invention, according to an exemplary embodiment.

FIG. 7 shows DGE-AUC analysis of AAV samples using three different cesium chloride densities, according to an exemplary embodiment.

FIG. 8 shows DGE-AUC analysis of AAV samples using three different rotor speeds, according to an exemplary embodiment.

FIG. 9 shows a comparison of DGE-AUC analysis of AAV samples containing either empty or empty and full capsids utilizing a cesium chloride gradient and speed of about 40000 rpm, according to an exemplary embodiment.

FIG. 10 shows a comparison of DGE-AUC analysis of AAV samples containing either empty or empty, partially filled and full capsids utilizing a cesium chloride gradient and speed of about 40000 rpm, according to an exemplary embodiment.

FIG. 11 shows a comparison of DGE-AUC analysis of AAV samples using different salts, according to an exemplary embodiment.

FIG. 12 shows A260/A280 ratios of empty and full capsids measured using DGE-AUC analysis utilizing a cesium sulphate gradient and speed of about 40000 rpm, according to an exemplary embodiment.

FIG. 13 illustrates the preparation of AAV samples in neat solution, diluted in placebo and affinity purified, or diluted in HEK293 transfection media and affinity purified, according to an exemplary embodiment.

FIG. 14 shows quantification of empty and full capsids by DGE-AUC analysis of samples in neat solution, diluted in placebo and affinity purified, or diluted in HEK transfection media and affinity purified utilizing a cesium sulphate gradient and speed of about 40000 rpm, according to an exemplary embodiment.

FIG. 15 shows DGE-AUC analysis of an affinity purified TFF pool utilizing a cesium sulfate gradient and speed of about 40000 rpm, according to an exemplary embodiment.

FIG. 16 shows DGE-AUC analysis of an affinity purified harvest pool utilizing a cesium sulfate gradient and speed of about 40000 rpm, according to an exemplary embodiment.

FIG. 17 illustrates an affinity purification process for crude AAV5 samples, according to an exemplary embodiment.

FIG. 18A shows a quantification of empty, partially filled and full capsids for a neat sample of AAV5 by DGE-AUC analysis utilizing a cesium sulphate gradient and speed of about 40000 rpm, according to an exemplary embodiment.

FIG. 18B shows a quantification of empty, partially filled and full capsids for an affinity purified AAV5 sample by DGE-AUC analysis utilizing a cesium sulphate gradient and speed of about 40000 rpm, according to an exemplary embodiment.

FIG. 18C shows a comparison of quantifications of empty, partially filled and full capsids for a neat AAV5 sample and an affinity purified AAV5 sample, according to an exemplary embodiment.

FIG. 19A shows a quantification of empty, partially filled and full capsids for a clarified cell lysate sample of AAV5 by DGE-AUC analysis utilizing a cesium sulphate gradient and speed of about 40000 rpm, according to an exemplary embodiment.

FIG. 19B shows quantification of empty, partially filled and full capsids fo a clarified cell lysate sample of AAV5, according to an exemplary embodiment.

### DETAILED DESCRIPTION

Adeno-associated viruses (AAVs) have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1-Unique N-Terminal Domain and Its Role in Capsid Trafficking, Journal of Virology, May, 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. The purity of AAVs is defined by several product-related impurities, including empty capsids, capsids containing partial or incorrect genomes, and aggregated or degraded capsid, as well as residual host cell proteins (HCP).

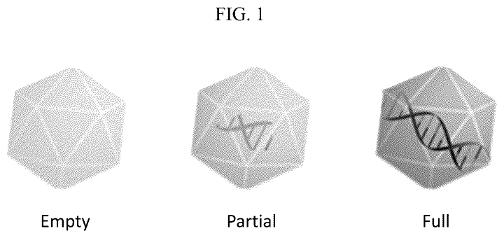

An inherent characteristic of the AAV manufacturing process is the production of empty capsids, i.e. capsids lacking any packaged transgene. Empty capsids are impurities that may result in an unwanted immune response and may reduce potency of the gene therapy drug. An empty capsid has a molecular weight of about ˜3750 kDa, while a full capsid with an about 4.7 kb single-stranded genome has a molecular weight of about ˜5100 kDa. In addition to empty capsids, a heterogeneous population of partially filled capsids, containing process-related impurities or a truncated transgene, may also be produced during manufacturing, as shown in FIG. 1. These impurities can lower the overall yield and therapeutic potential of the gene therapy product (full capsids). Aggregated or degraded capsids, which may present as low or high molecular weight species in a sample, also must be monitored, as they may reduce viral titer and efficacy while increasing the load of potentially immunogenic viral protein. The ratio of empty-full capsids, empty-partially filled-full capsids, the presence of degraded or aggregated capsids, and the presence of low molecular weight species or high molecular weight species may be critical quality attributes (CQA) for a viral vector gene therapy product.

The purity of AAV products is critical for improved transduction and efficacy of the product. Therefore, it is essential to have an analytical method that can provide quantitative information about the empty/partially filled/full distribution (also called “capsid content”) of AAV capsids at an early stage of an AAV production cycle. Having this information early in AAV manufacturing and processing could help determine the batch quality and save time and cost involved in subsequent purification steps. It can also help in the development of new cell lines for recombinant AAV production to determine whether the cells are able to produce full capsids.

AAVs are the primary delivery vehicle for gene therapy applications. To ensure efficient AAV production by upstream manufacturing, and high overall yields, it is crucial to determine the quality of AAVs being produced prior to intensive downstream processing steps. AAV downstream processing may involve multiple steps like harvesting by cell lysis, clarification of lysate, concentration and buffer exchange (TFF), affinity capture, additional chromatography steps such as anion exchange chromatography (AEX), and concentration by UF/DF.

Several analytical methods exist in the literature for quantitation of capsid content in pure AAV samples, including sedimentation velocity-analytical ultracentrifugation (SV-AUC), anion exchange chromatography and transmission electron microscopy. However, no existing analytical method can provide quantitative information on capsid content in a crude AAV sample (for example, harvest pool and/or TFF pool) obtained during the initial steps of downstream AAV processing. In order to overcome this issue, a standardized and effective method for quantifying capsid content in AAV samples early in the downstream purification process has been developed.

The present application provides methods to characterize AAV capsid content. In some exemplary embodiments, the AAVs are affinity purified and characterized by density gradient equilibrium analytical ultracentrifugation (DGE-AUC), allowing for separation of empty, partially filled and full AAVs.

This disclosure provides methods to satisfy the aforementioned demands by providing methods to identify the capsid content in biopharmaceutical products to mitigate safety risks. Exemplary embodiments disclosed herein satisfy the aforementioned demands and the long-felt needs.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Muller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” refers to a mixture of molecules that comprises at least one viral particle, such as an AAV capsid particle, or empty or partially filled or full viral capsid, that is subjected to manipulation in accordance with the methods of the invention, including, for example, separating, analyzing, extracting, concentrating, profiling and the like.

As used herein, the term “impurity” can include any undesirable protein or nucleic acid present in a sample or biopharmaceutical product, or an undesirable modified form of a biopharmaceutical product. Impurities can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, the term “crude sample” can include any sample in the manufacturing and purification process for a viral vector, for example an AAV vector, which has not been substantially enriched for the viral vector. For example, a crude sample may be a harvest pool or tangential flow filtration pool.

As used herein, the term “vector” refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant AAV vector (rAAV vector)” refers to a polynucleotide vector including one or more heterologous sequences (i.e., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins).

A “capsid” is the protein shell of a virus, which encloses the genetic material. Three viral capsid proteins, VP1, VP2 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

A “viral particle” refers to a particle composed of at least one viral capsid and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roseolovirus, Lymphocryptovirus, Macavirus, Percavirus, and Rhadinovirus.

As used herein, the term “analytical ultracentrifugation” or “AUC” refers to a process combining centrifugation with optical monitoring systems to characterize the size and shape of samples in their native state under biological conditions. Non-limiting examples of information that can be obtained include determination of molecular weight and protein degradation or aggregation. Non-limiting examples of factors affecting the sedimentation of analytes include rotor speed, temperature, distance between the rotor center and the sample, solvent and time.

As used herein, the term “density gradient equilibrium” or “DGE” refers to a process to separate a mixture of macromolecules based on their buoyant density utilizing analytical ultracentrifugation. A homogeneous solution of a gradient-forming salt and a macromolecular system, for example an AAV sample, is formed by mixing a salt solution with the sample of interest. The homogeneous solution is then subjected to analytical ultracentrifugation, which generates a density gradient of the salt. After centrifugation to equilibrium in a density gradient, macromolecules sediment into a band, wherein the solution density at this position is the same as the buoyant density of the macromolecule species. A solution containing macromolecules of interest may be subjected to ultracentrifugation employing a salt gradient. Ultracentrifugation of the mixture is performed until equilibrium is attained. At equilibrium, macromolecules will form a band where the density of the macromolecule is the same as the density of the salt. Denser macromolecules (for example, a full capsid) will accumulate towards the bottom of the cell (sample holder) whereas lighter macromolecules (for example, an empty capsid) will form a band closer to the top of the cell. Non-limiting examples of gradient-forming salts for use in such a salt gradient include cesium chloride, cesium sulfate, cesium formate, cesium acetate, cesium iodide, cesium selenite, potassium bromide, rubidium bromide, rubidium chloride, cesium TFA, sodium iothalamate, or cesium bromide.

One advantage of DGE-AUC is that it is size-independent, because molecules are separated based on relative density, not size. Another advantage is its sensitivity: samples are effectively concentrated as they migrate through the density gradient to form a band. This is very effective for low concentration analytes, for example low titer AAV vector samples. In particular, as described further below, the sensitivity of DGE-AUC allows for accurate quantitation of capsid content in AAVs extracted from crude samples, which have a titer too low for conventional quantitative assays. Furthermore, compared to alternative methods such as sedimentation velocity AUC (SV-AUC), DGE-AUC offers simple analysis, since the raw data may be analyzed without the need for complex computations.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

As used herein, the term “affinity capture” or “affinity purification” is a process in which a biological/chemical mixture carried by a liquid can be separated into components as a result of their chemical properties as they flow through, or bind to, a solid substrate or solid support. Analytes separated using affinity capture will bind to the solid support based on their affinity, and will elute from the solid support under proper conditions. The solid support may be conjugated to an affinity ligand that specifically binds to the analyte(s) of interest; for example, an affinity ligand may be an antigen-binding protein such as an antibody, variant thereof or fragment thereof. A solid support may also be conjugated to an affinity ligand that specifically binds to a complementary moiety that can be used to label an analyte; for example, an affinity ligand or its complementary moiety may be biotin, avidin or streptavidin. In some aspects, an affinity resin is used to enrich viral vectors in a sample, for example AAV vectors. In some aspects, an affinity resin may be POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, any other resin comprising an affinity ligand for a viral vector, or a combination thereof.

As used herein, the term “small-scale” includes experimental volumes ranging from 1 μL to 10 mL. In some exemplary embodiments, less than about 100 μL was utilized for a purified AAV sample. In yet another exemplary embodiment, less than about 1 mL was utilized for a TFF pool sample. In yet another exemplary embodiment, 10 mL or less was utilized for a harvest pool sample.

As used herein, the term “medium throughput” denotes an assay with an intermediate level of testing capacity, capable of analyzing multiple biological samples simultaneously or in parallel. In some aspects, the methods of the present invention allow for greater than 5, greater than 10, greater than 20, greater than 30, greater than 40, or greater than 50 samples to be affinity purified, enriched, and/or characterized simultaneously.

“Contacting,” as used herein, includes bringing together at least two substances in solution or solid phase, for example contacting a stationary phase of a chromatography material with a sample, such as a sample comprising viral particles.

It is understood that the present invention is not limited to any of the aforesaid protein(s), therapeutic protein(s), antibody(s), recombinant protein(s), protein pharmaceutical product(s), sample(s), virus(es), serotype(s), vector(s), salt(s), chromatographic method(s), pH range(s) or value(s), temperature(s), or concentration(s), and any protein(s), therapeutic protein(s), antibody(s), recombinant protein(s), protein pharmaceutical product(s), sample(s), virus(es), serotype(s), vector(s), salt(s), chromatographic method(s), pH, temperature(s), or concentration(s) can be selected by any suitable means.

The present invention will be more fully understood by reference to the following examples. They should not, however, be construed as limiting the scope of the invention.

### EXAMPLES

### Example 1. DGE-AUC Method for Capsid Content Analysis

Current methods for AAV vector production and purification include lysis of cells that recombinantly produce an AAV vector, harvesting of cell culture components and removal of cellular material, tangential flow filtration (TFF) for removal of low molecular weight impurities, followed by additional chromatography steps such as anion exchange (AEX) chromatography for continued purification and exchange of buffers, followed by concentration by ultrafiltration and diafiltration (UF/DF), resulting in concentrated AAV particles. However, there are several challenges and shortcomings with current methods for AAV purification. Some of the challenges and shortcomings include, for example, the co-purification of empty, partially filled and full capsids and the presence of impurities in the final product.

The co-purification of empty and partially filled capsids is a major disadvantage. These impurities can result in unwanted immune responses and may reduce the potency of the gene therapy drug. Conventionally, sedimentation velocity analytical ultracentrifugation (SV-AUC) has been used to determine the empty/partially filled/full ratio. However, this method is material intensive and is based on separation of molecules based on weight. An alternative approach involves density gradient equilibrium analytical ultracentrifugation (DGE-AUC), wherein the AAV sample is contacted to a salt solution of a gradient-forming salt to form a homogeneous solution, followed by analytical ultracentrifugation to separate macromolecules based on density.

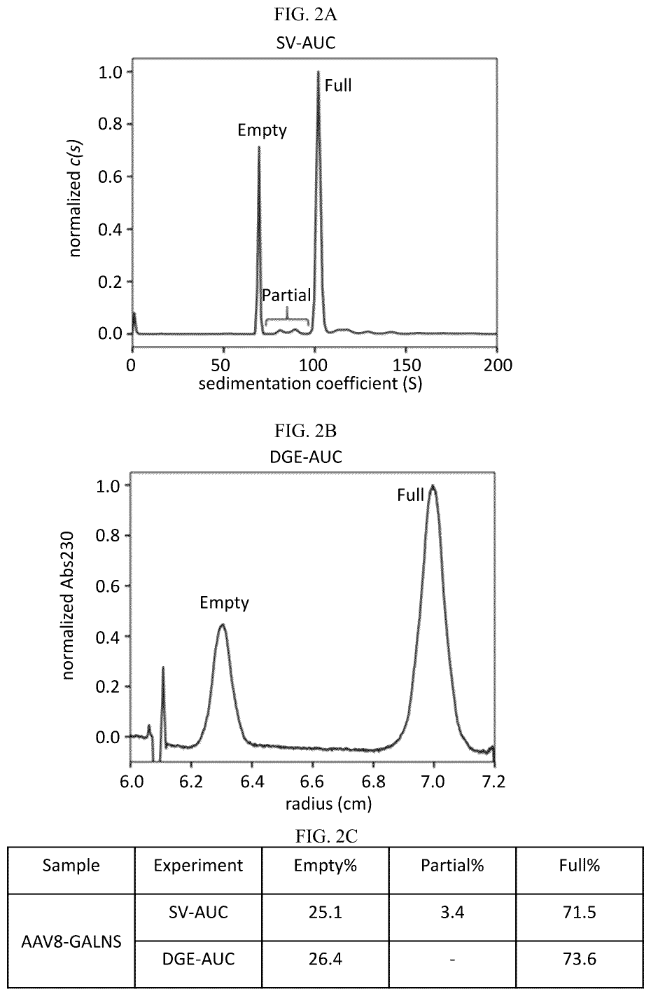

The DGE-AUC method was compared to the SV-AUC method using a purified sample of an AAV8 vector. FIG. 2A shows the separation of full, empty and partial capsids after conventional SV-AUC. Analysis using a 1.34 g/mL cesium chloride salt solution for DGE-AUC shows concordant separation of full and empty capsids, as shown in FIG. 2B. The quantification of the measured values for full, partial and empty capsids is shown in FIG. 2C. The percent of empty and full capsids identified in the analysis of the two methods was in good agreement with each other, with SV-AUC and DGE-AUC respectively measuring 25.1% and 26.4% empty capsids, and 71.5% and 73.6% full capsids.

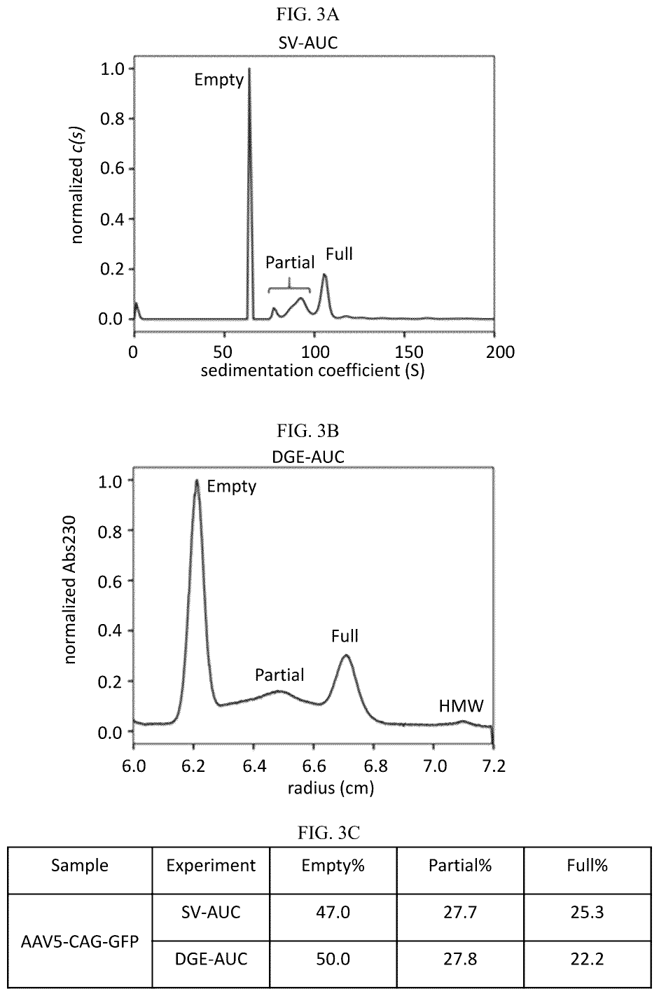

A comparison of SV-AUC and DGE-AUC methods using a purified sample of AAV5 was subsequently tested. FIG. 3A shows the separation of full, empty and partial capsids after conventional SV-AUC. Analysis using a 1.34 g/mL cesium sulfate salt solution for DGE-AUC shows concordant separation of full, partially filled, and empty capsids, as shown in FIG. 3B. FIG. 3C compares the quantification of the measured values using SV-AUC and DGE-AUC. The percent of empty, partially filled, and full capsids identified in the analysis of the two methods was in good agreement with each other, with SV-AUC and DGE-AUC respectively measuring 47.0% and 50.0% empty capsids, 27.7% and 27.8% partially filled capsids, and 25.3% and 22.2% full capsids. These results confirm that the DGE-AUC method used for separation and quantification of empty, partially filled, and full capsids is a valuable method.

### Example 2. DGE-AUC Method for In-Process Production Samples

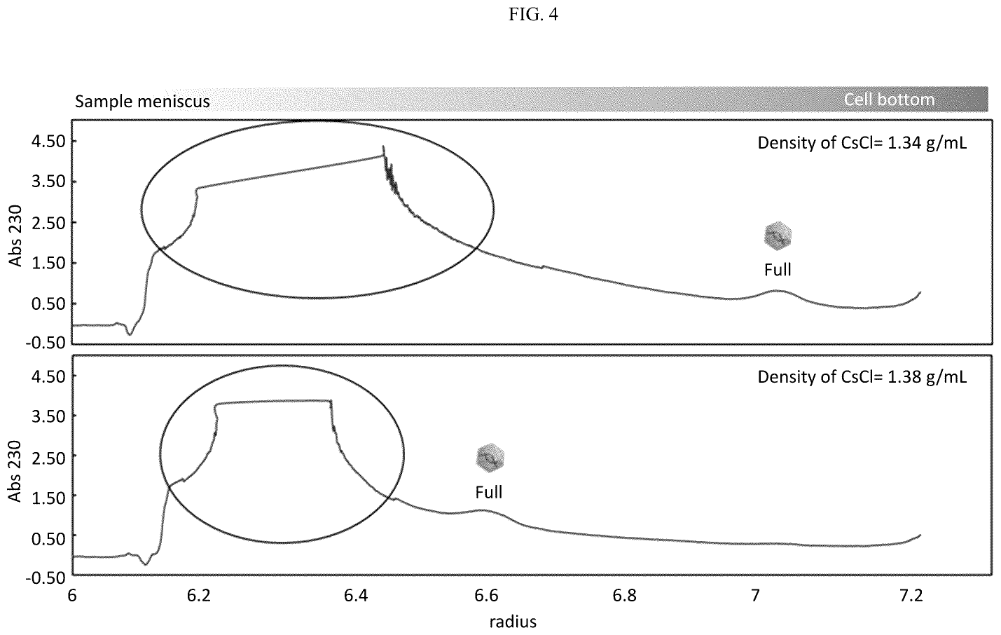

Based on the effectiveness of the DGE-AUC method as shown in Example 1, the method was further tested on in-process crude samples. The TFF pool of an AAV vector production process was tested to quantify full, empty and/or partially filled capsid content. However, a significant amount of background signal observed in the analysis prevented the quantification of capsid content, as shown in FIG. 4.

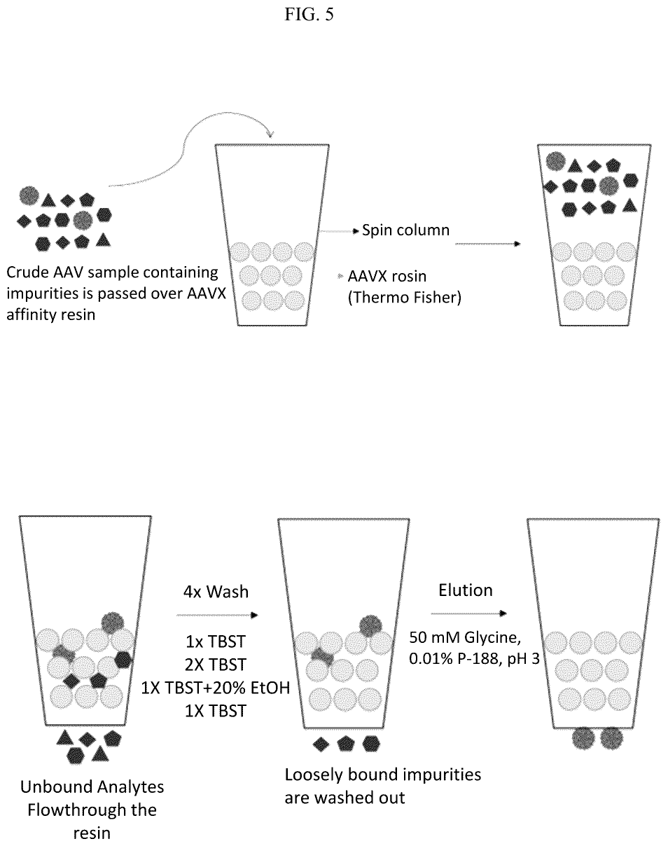

In order to overcome the challenge of capsid content analysis in crude samples, a method was developed including affinity purification prior to DGE-AUC analysis. A small-scale affinity purification was developed to determine if removal of impurities causing the background would provide more accurate results, as shown in FIG. 5. The affinity purification strategy includes obtaining a crude AAV vector sample containing impurities and adding it to a spin column comprising an affinity resin against the AAV, for example POROS™ CaptureSelect™ AAVX Affinity Resin or AVB Sepharose™. Unbound analytes were allowed to flow through the resin. The resin was then subjected to four wash steps to remove loosely bound impurities. The purified AAV was then eluted and subjected to DGE-AUC analysis. Affinity purification of 500-600 μL of crude TFF pool yielded enough purified AAV vector to run duplicate analyses.

### Example 3. Optimization of the DGE-AUC Method

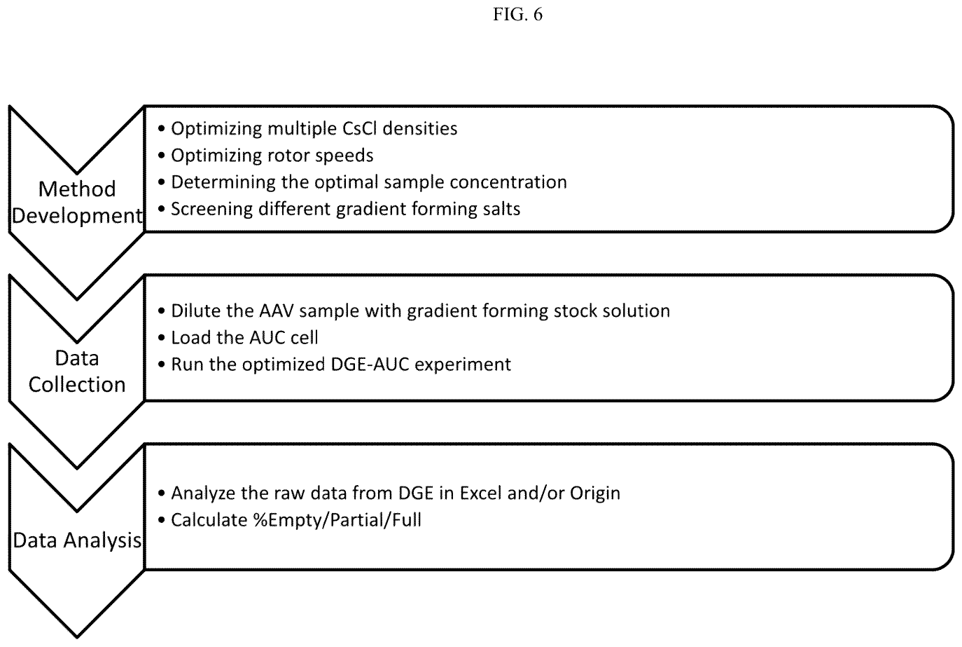

In order to optimize separation and quantification of empty, partially filled, and full capsids, a variety of DGE-AUC parameters were tested. The method was conducted with varying salt density, rotor speeds, sample concentration, and different gradient forming salts. A workflow of parameters tested, as well as an overview of exemplary data collection and data analysis steps of the method of the present invention, is shown in FIG. 6.

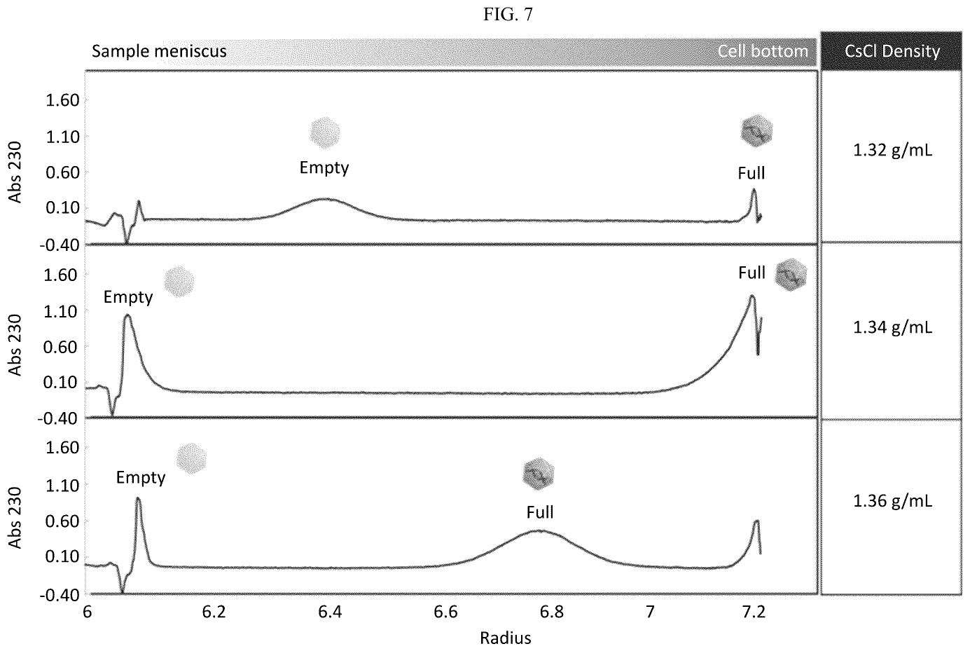

The initial density of cesium chloride used in the salt solution for DGE-AUC has a profound impact on the separation of empty and full capsids, as shown in FIG. 7. At lower cesium chloride densities, for example about 1.32 g/mL, the full capsid was very dense compared to the salt gradient and settled at the bottom of the cell. At higher cesium chloride densities, for example about 1.36 g/mL, the empty capsid was very light compared to the salt gradient and floated on the top of the cell. Maximum separation of empty and full capsids was seen at a cesium chloride density of about 1.34 g/mL.

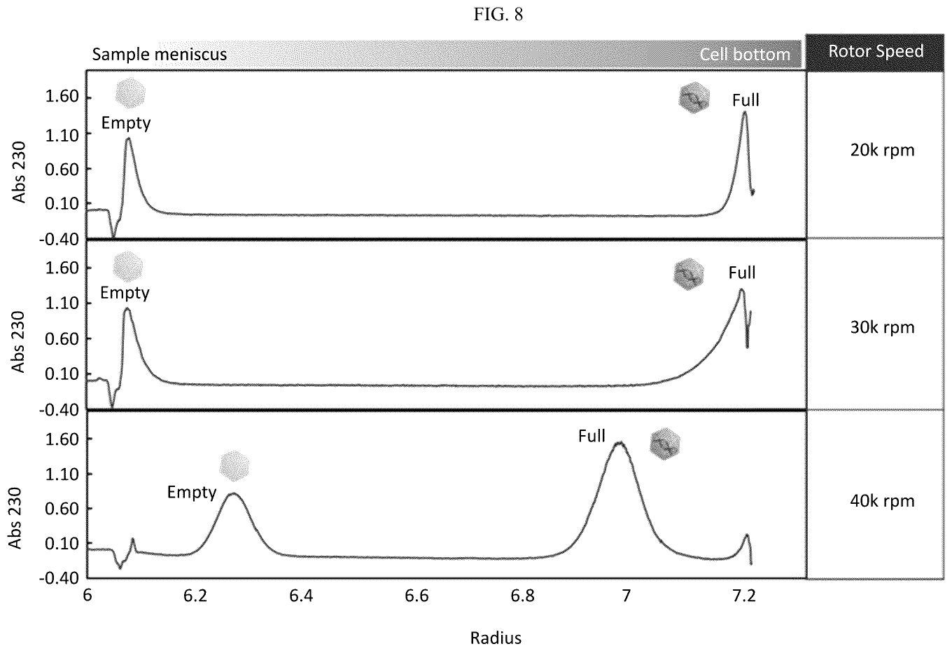

The effect of rotor speed on the separation of empty and full capsids is shown in FIG. 8. At lower rotor speeds, for example about 20 k rpm, a shallow gradient is formed, leading to a large separation of the empty and full capsids in the sample. At higher rotor speeds, for example about 40 k rpm, a steeper gradient is formed, leading to optimal separation of empty and full capsids.

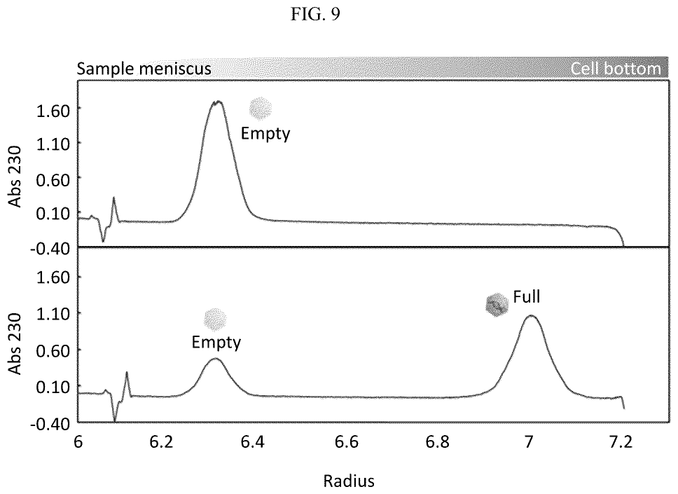

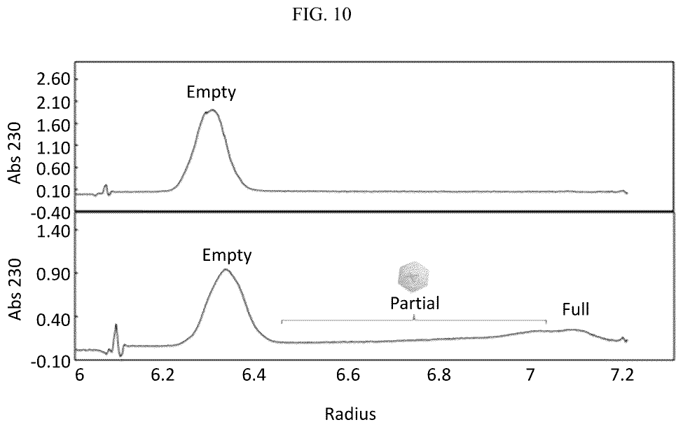

The cesium chloride and rotor speed conditions described above were effective for separating empty and full capsids in a sample. A comparison of a capsid content analysis of two AAV8 samples, one empty and one full, using the optimized parameters is shown in FIG. 9. The empty capsid bands at a radius of about 6.3 cm (top panel) and the full capsid bands at a radius of about 7.0 cm (bottom panel). The same conditions were then applied for a sample known to contain a high percentage of partially filled capsids. A comparison of two AAV5 samples, one empty and one full, is shown in FIG. 10. The empty AAV sample shows an empty capsid band at a radius of about 6.3 cm, consistent with the AAV8 sample in FIG. 9. However, partially filled capsids were not adequately separated from full capsids using these conditions, as shown in the bottom panel. Therefore, this AAV5 lot was used to further optimize DGE-AUC conditions to improve the resolution between partially filled and full capsids.

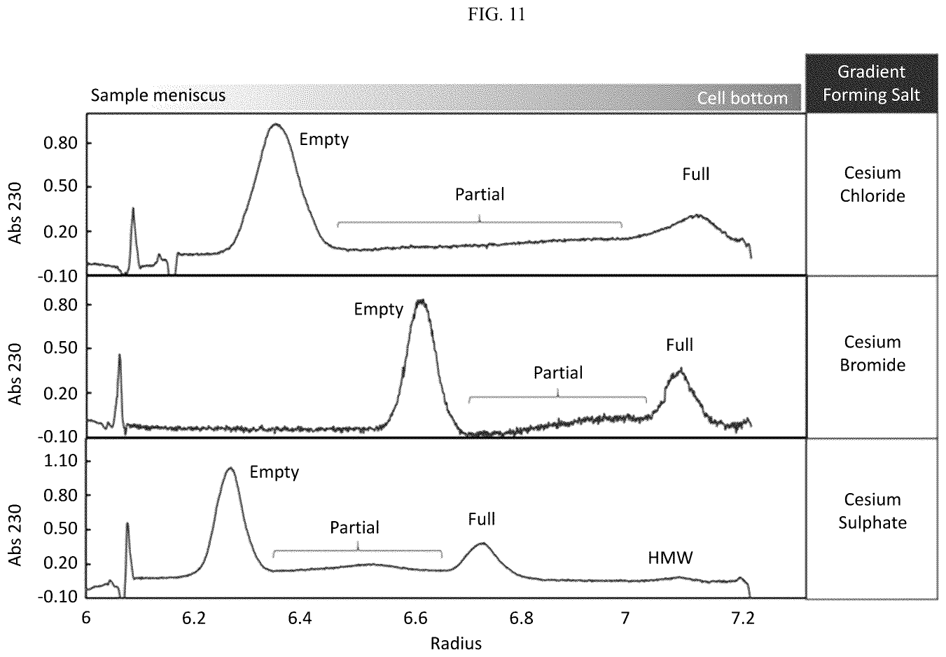

To improve the separation between full and partially filled capsids, different salts were tested, using the same AAV5 sample for optimization. Three salts, cesium chloride, cesium bromide and cesium sulfate, were compared as shown in FIG. 11. Cesium chloride provided the best separation between empty and full capsids, but didn't separate partially filled and full capsids sufficiently. Cesium bromide demonstrated good separation between empty, partially filled, and full, however a lot of noise was observed in the UV trace. Cesium sulfate demonstrated the best separation between empty, partially filled, and full capsids. Additionally, high molecular weight species could be observed within the same run.

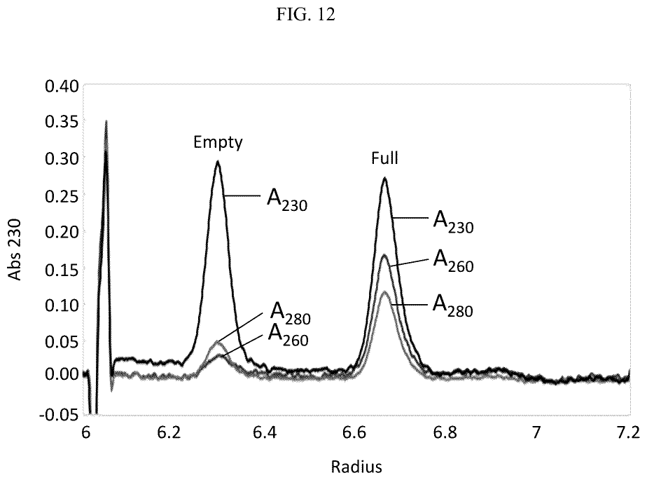

The ability to use multiple UV absorbance measurements during the DGE-AUC analysis allows for the calculation of the A₂₆₀/A₂₈₀ ratio to determine the capsid content for the empty and full capsids. A₂₆₀/A₂₈₀ ratio is a measure of nucleic acid purity, and therefore the measured A₂₆₀/A₂₈₀ ratio would be expected to be low for empty capsids and high for full capsids. FIG. 12 shows an overlay of measured A230, A260 and A280 signals, as well as the A₂₆₀/A₂₈₀ ratio for the empty and full capsids. An empty capsid has a typical A₂₆₀/A₂₈₀ value of about 0.5, while a full capsid has a typical A₂₆₀/A₂₈₀ value of about 1.4. This result validates the identification of the DGE-AUC peaks as corresponding to empty or full capsids.

### Example 4. Validation of the Affinity DGE-AUC Method

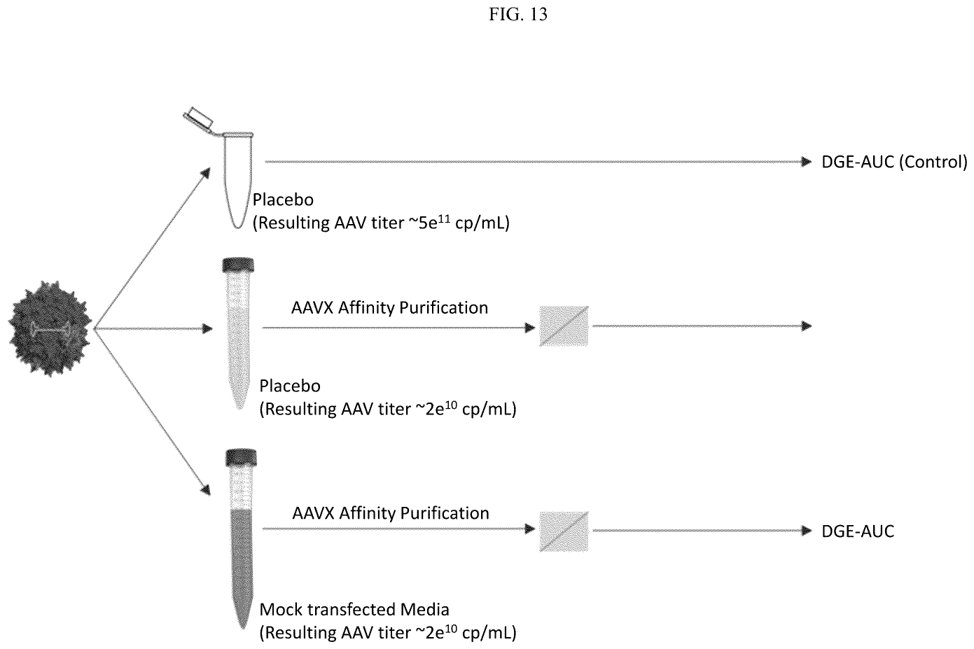

The efficiency and accuracy of the affinity purification and DGE-AUC method of the present invention was confirmed using an AAV8 sample diluted in different solutions. As depicted in FIG. 13, three separate AAV8 vector samples were prepared: (1) isolated AAV8 with a titer of about 5×10¹¹ capsids/mL (cp/mL), (2) the AAV of (1) diluted in a placebo solution to a titer of about 2×10¹⁰ cp/mL and affinity purified, and (3) the AAV of (1) diluted in mock transfected media to a titer of about 2×10¹⁰ cp/mL and affinity purified.

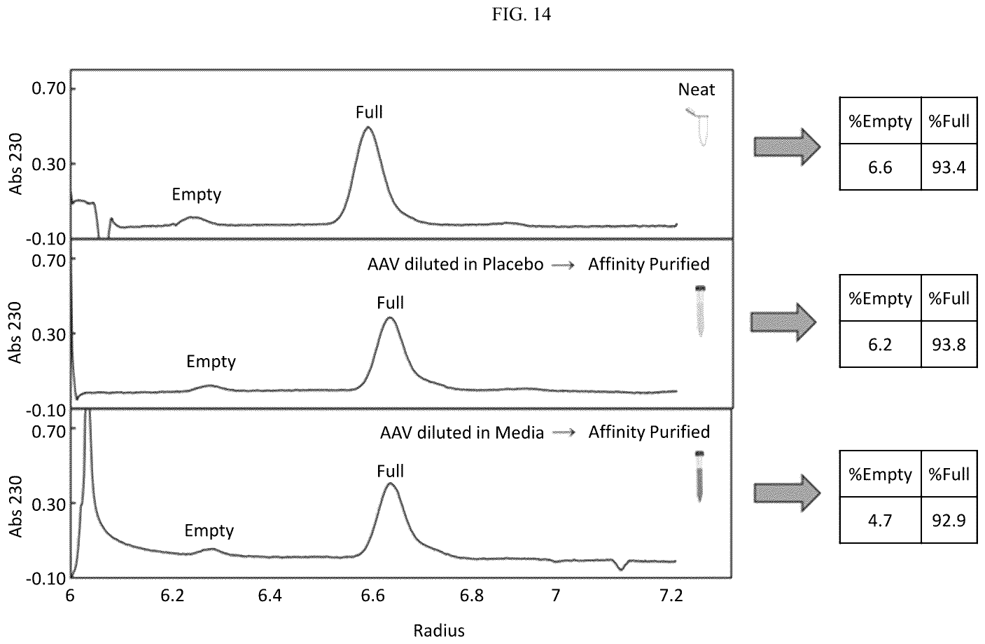

The three affinity purified samples were subjected to DGE-AUC analysis and compared, as shown in FIG. 14. The affinity purification step was found to be efficient in both the AAV8 diluted in placebo and the AAV8 diluted in media, with a recovery of 74% and 77% respectively relative to the non-diluted AAV8 sample. Additionally, the quantification of empty and full capsids was consistent across the samples, validating that affinity purification and DGE-AUC of even very low concentration and complex AAV samples produced a consistent and sensitive measurement of capsid content.

### Example 5. Capsid Content Analysis in Crude Samples Using the Affinity DGE-AUC Method

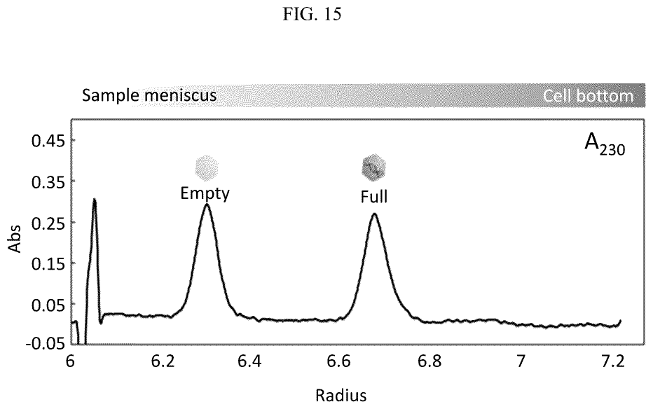

The optimized affinity DGE-AUC method using 1.33 g/mL cesium sulfate was applied to crude AAV samples from an in-house production process. Compared to a DGE-AUC method without affinity purification as shown in FIG. 4, the novel affinity DGE-AUC method was capable of removing background from a TFF pool and providing excellent separation and quantitation of empty and full species, as shown in FIG. 15.

As described above, a benefit of capsid content analysis of crude samples is the early identification of any quality issues in a production process. Assessment of capsid content at the earliest possible stage would save time and resources on subsequent steps. DGE-AUC analysis of capsids as early in a production process as a TFF pool has not previously been shown due at least in part to the low AAV titer and complexity of cell culture components present in the sample. The earliest sample in a production process that may be analyzed for capsid content is the step preceding TFF, the harvest pool, which is about ten times less concentrated than the TFF pool. In order to quantify capsid content as early as possible in an AAV vector production process, the affinity DGE-AUC method was applied to a harvest pool.

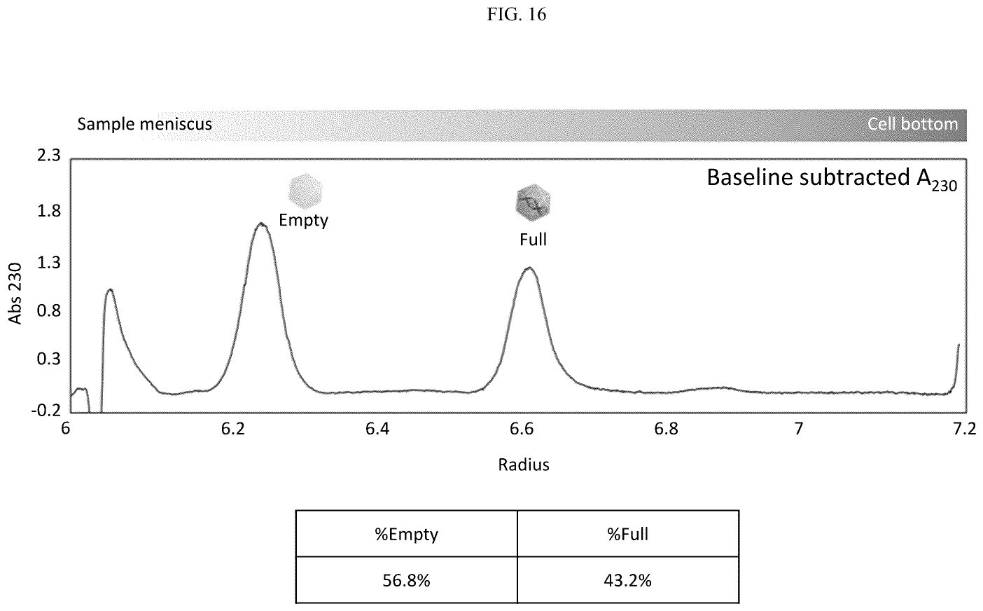

Capsid content analysis of a harvest pool using affinity DGE-AUC is shown in FIG. 16. About 10 mL of harvest pool was sufficient for affinity DGE-AUC analysis. Using the affinity DGE-AUC method of the present invention, full and empty capsids were well separated and quantification of full (43.2%) and empty (56.8%) species was observed, allowing for a sensitive and early characterization of capsid content in an AAV vector production process.

### Example 6. Affinity Purification and DGE-AUC Characterization of AAV5

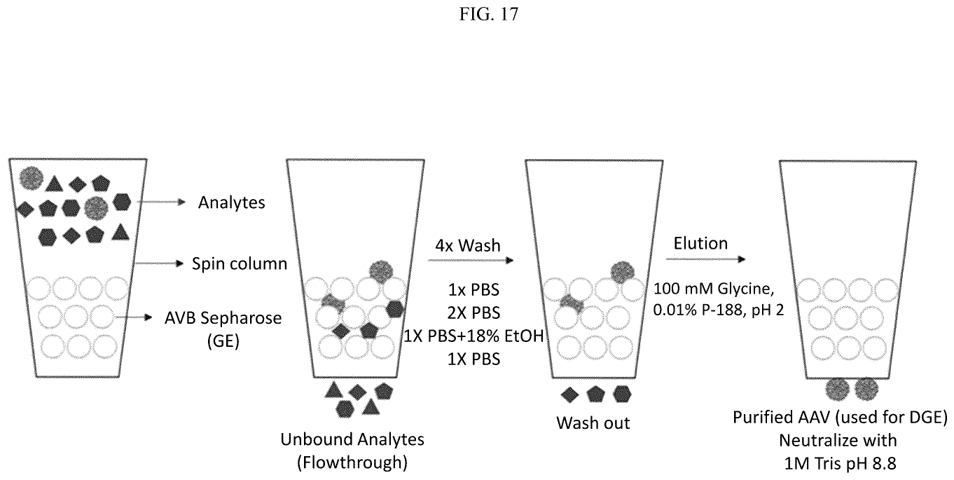

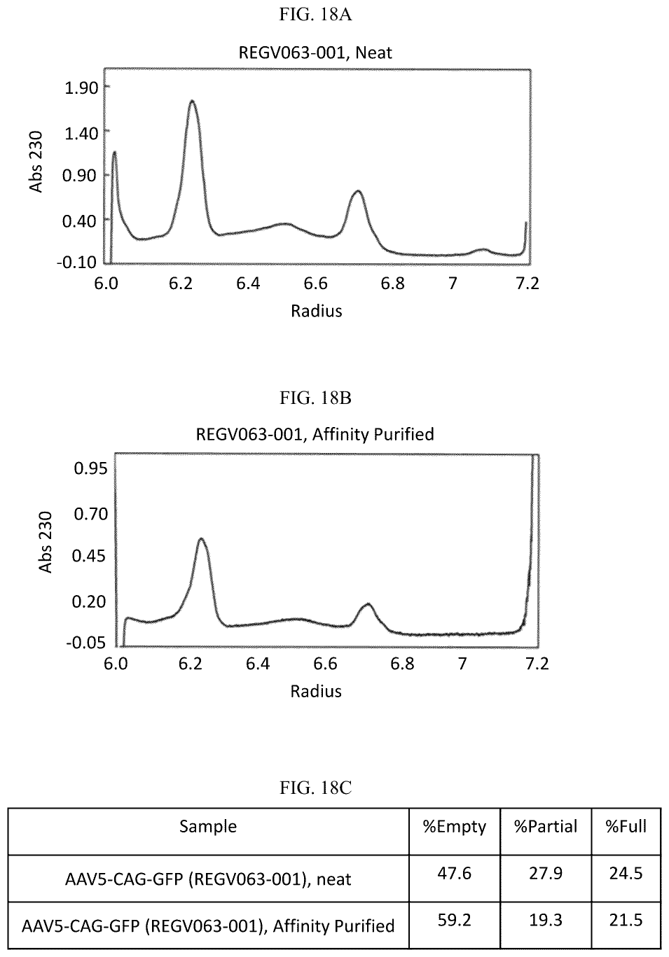

A small-scale affinity purification scheme for AAV5 is illustrated in FIG. 17. REGV063-001 neat sample is REGV063-001 diluted in placebo buffer and subsequently run on DGE-AUC. REGV063-001 affinity purified sample is REGV063-001diluted in a large excess of placebo buffer and purified using the affinity method illustrated in FIG. 17. The purified sample was subsequently run on DGE-AUC. FIGS. 18A-C show the DGE-AUC analysis of neat REGV063-001 having an empty/partial/full ratio of about 48/28/24%, whereas the ratio is about 59/19/21% for the affinity purified sample indicating a minor reduction in % full after affinity purification.

### Example 7. Purification and Characterization of an AAV5 Sample from a Cell Lysate

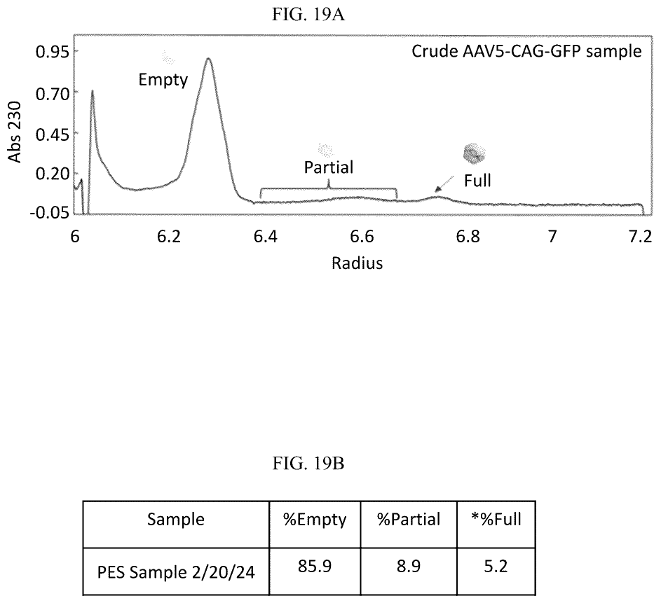

A cell lysate of AAV5-CAG-GFP (PES Sample 2/20/24, which is a cell lysate containing AAV5-CAG-GFP, expressed following HEK293 stable cell line transfection) was subjected to small-scale affinity purification to clear the background from the crude sample. DGE-AUC was performed on the purified sample which showed about 85% empty, about 10% partial and about 5% full capsids (see FIGS. 19A and B). The DGE-AUC analysis of a clarified cell lysate indicates that the method can effectively determine empty/partial/full species of AAV's, thus making it a useful method to guide the development of stable cell lines.

This disclosure sets forth novel methods for characterizing viral vector samples, in particular the capsid content of AAV vectors, using affinity purification and DGE-AUC. A small-scale affinity purification strategy was developed that is quick and cost-effective. The affinity purification step using an affinity resin such as, POROS™ CaptureSelect™ AAVX Affinity Resin or AVB Sepharose™, does not affect the empty/full ratio. The use of DGE-AUC allows for the sensitive quantitation of very low titer samples that cannot be analyzed using a conventional SV-AUC analysis, making it uniquely synergistic with the low titer eluate from the small-scale affinity purification step. AAV vectors extracted directly from transfection media could be successfully characterized. Therefore, the capsid content in crude samples early in a production process can be assessed, shedding light on an important critical quality attribute in the gene therapy field. Assessing the quality of an AAV batch early in the process cycle can help in making appropriate decisions on drug development, reducing time and expenses associated with later purification steps. Furthermore, obtaining this information could help in the development of new cell lines for recombinant AAV production by determining their capacity to generate full AAV capsids.

1. A method of characterizing at least one AAV vector in a crude sample or in a sample containing at least one impurity, comprising:
(a) subjecting a sample containing at least one AAV vector to affinity purification to produce an enriched sample;
(b) contacting said enriched sample to a salt solution to form a homogeneous solution; and
(c) subjecting said homogeneous solution to density gradient equilibrium analytical ultracentrifugation to characterize the at least one AAV vector.

2. The method of claim 1, wherein said affinity purification comprises affinity chromatography.

3. The method of claim 1, wherein the affinity purification is a small-scale affinity purification.

4. The method of claim 1, wherein the affinity purification occurs in a medium throughput manner.

5. The method of claim 1, wherein said affinity purification comprises contacting said sample to a resin conjugated to an affinity ligand.

6. The method of claim 5, wherein said affinity ligand comprises an antigen-binding protein, an antibody, a variant thereof, or a fragment thereof.

7. The method of claim 6, wherein said resin is POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, or a combination thereof.

8. The method of claim 5, further comprising subjecting said resin to at least one wash step, at least two wash steps, at least three wash steps, or at least four wash steps.

9. The method of claim 8, wherein a wash buffer used in the wash step(s) comprises about 1×TBS, about 2×TBS, about 1×TBS with 20% ETOH, about 1×PBS, about 2×PBS, about 1×PBS with 18% EtOH, or combinations thereof.

10. The method of claim 1, wherein said affinity purification comprises an elution step.

11. The method of claim 10, wherein said elution step includes an elution buffer, optionally wherein said elution buffer comprises glycine, poloxamer 188, or a combination thereof.

12. The method of claim 10, wherein said elution buffer comprises about 50 mM glycine and about 0.01% poloxamer 188 at a pH of about 3 or about 100 mM glycine and about 0.01% poloxamer 188 at a pH of about 2.

13. The method of claim 1, wherein said salt solution includes cesium chloride, cesium sulfate, cesium bromide, cesium formate, cesium acetate, cesium iodide, cesium selenite, potassium bromide, rubidium bromide, rubidium chloride, cesium TFA, sodium iothalamate, or a combination thereof.

14. The method of claim 1, wherein a concentration of salt in said salt solution is about 1.30 g/mL to about 1.40 g/mL or about 1.32 g/mL to about 1.36 g/mL.

15. The method of claim 14, wherein said salt concentration is about 1.34 g/mL.

16. The method of claim 1, wherein a rotor speed of the analytical ultracentrifuge is from 10 k to 60 k rpm.

17. The method of claim 16, wherein said rotor speed is about 40 k rpm.

18. The method of claim 1, wherein said at least one AAV vector comprises a serotype selected from a group consisting of AAV1, AAV2, AAV3, AAV4, AAV5, AAV6, AAV7, AAV8, AAV9, AAV10, AAV11, AAV12, variations thereof and combinations thereof.

19. The method of claim 1, wherein said sample is a crude sample.

20. The method of claim 1, wherein said sample comprises a harvest pool or a tangential flow filtration pool.

21. The method of claim 1, wherein a concentration of said at least one AAV vector in said sample is from 1×1010 to 1×1012 cp/mL.

22. The method of claim 1, further comprising quantifying empty capsids, partially filled capsids, and/or full capsids of said at least one AAV vector.

23. The method of claim 1, further comprising quantifying a ratio of absorbance at 260 nm to absorbance at 280 nm wavelengths of AAV vector species separated by density gradient equilibrium analytical ultracentrifugation.

24. The method of claim 1, further comprising detecting and/or quantifying high molecular weight species.

25.-47. (canceled)

48. A method for characterizing a viral vector, comprising:
(a) subjecting cells that recombinantly produce a viral vector to lysis to form a lysed sample;
(b) subjecting said lysed sample to filtration to form a harvest pool;
(c) subjecting said harvest pool to affinity purification to form an enriched viral vector sample; and
(d) subjecting said enriched viral vector sample to density gradient equilibrium analytical ultracentrifugation to characterize said viral vector.

49. The method of claim 48, further comprising subjecting said harvest pool to tangential flow filtration prior to step (c).

50. The method of claim 48, wherein the affinity purification is a small-scale affinity purification.

51. The method of claim 48, wherein the affinity purification occurs in a medium throughput manner.

52. The method of claim 48, wherein said affinity purification comprises affinity chromatography.

53. The method of claim 48, wherein said affinity purification comprises contacting said sample to a resin conjugated to an affinity ligand.

54. The method of claim 53, wherein said affinity ligand comprises an antigen-binding protein, an antibody, a variant thereof, or a fragment thereof.

55. The method of claim 54, wherein said resin is POROS™ CaptureSelect™ AAVX Affinity Resin, POROS™ CaptureSelect™ AAV8 Affinity Resin, POROS™ CaptureSelect™ AAV9 Affinity Resin, AVB Sepharose™, or a combination thereof.

56. The method of claim 53, further comprising subjecting said resin to at least one wash step, at least two wash steps, at least three wash steps or at least four wash steps.

57. The method of claim 56, wherein a wash buffer used in the wash step(s) comprises about 1×TBS, about 2×TBS, about 1×TBS with 20% ETOH, about 1×PBS, about 2×PBS, about 1×PBS with 18% EtOH, or combinations thereof.

58. The method of claim 48, wherein said affinity purification comprises an elution step.

59. The method of claim 58, wherein said elution step includes an elution buffer, optionally wherein said elution buffer comprises glycine, poloxamer 188, or a combination thereof.

60. The method of claim 58, wherein said elution buffer comprises about 50 mM glycine and about 0.01% poloxamer 188 at a pH of about 3 or about 100 mM glycine and about 0.01% poloxamer 188 at a pH of about 2.

61. The method of claim 48, further comprising contacting said enriched viral vector sample to a salt solution to form a homogeneous mixture prior to step (d).

62. The method of claim 61, wherein said salt solution includes cesium chloride, cesium sulfate, cesium bromide, cesium formate, cesium acetate, cesium iodide, cesium selenite, potassium bromide, rubidium bromide, rubidium chloride, cesium TFA, sodium iothalamate, or a combination thereof.

63. The method of claim 62, wherein a concentration of salt in said salt solution is about 1.30 g/mL to about 1.40 g/mL or about 1.32 g/mL to about 1.36 g/mL.

64. The method of claim 63, wherein said salt concentration is about 1.34 g/mL.

65. The method of claim 48, wherein a rotor speed of the analytical ultracentrifuge is from 10 k to 60 k rpm.

66. The method of claim 65, wherein in said rotor speed is about 40 k rpm.

67. The method of claim 48, wherein said viral vector comprises an AAV vector.

68. The method of claim 67, wherein said AAV vector comprises a serotype selected from a group consisting of AAV1, AAV2, AAV3, AAV4, AAV5, AAV6, AAV7, AAV8, AAV9, AAV10, AAV 11, AAV12, variations thereof and combinations thereof.

69. The method of claim 48, wherein a concentration of said at least one viral vector in said sample is from 1×1010 to 1×1012 cp/mL.

70. The method of claim 48, further comprising quantifying empty capsids, partially filled capsids, and/or full capsids of said at least one viral vector.

71. The method of claim 48, further comprising quantifying a ratio of absorbance at 260 nm to absorbance at 280 nm wavelengths of viral vector species separated by density gradient equilibrium analytical ultracentrifugation.

72. The method of claim 52, further comprising detecting and/or quantifying high molecular weight species.

73. The method of claim 48, wherein said cells are HEK293, HeLa, or Chinese Hamster Ovary (CHO) cells.

74. The method of claim 73, wherein said cells are HEK293 cells.

75. A method for quantifying the capsid content of a viral vector, comprising:
(a) subjecting a sample including a viral vector to affinity purification to form an enriched sample; and
(b) subjecting said enriched sample to density gradient equilibrium analytical ultracentrifugation to quantify the capsid content of said viral vector.

76. A method for characterizing critical quality attributes of a viral vector, comprising:
(a) subjecting a sample including a viral vector to affinity purification to form an enriched sample; and
(b) subjecting said enriched sample to density gradient equilibrium analytical ultracentrifugation to characterize critical quality attributes of said viral vector.

77. The method of claim 76, wherein said critical quality attributes include the empty/full ratio of said viral vector, the empty/partially filled/full ratio of said viral vector, low molecular weight species of said viral vector, and/or high molecular weight species of said viral vector.
